(19) United States
(12) Patent Application Publication
(10) Pub. No.: US 2024/0018152 A1
(43) Pub. Date: Jan. 18, 2024

(54) NOVEL PROCESS FOR THE PREPARATION OF (S)-7-(1-ACRYLOYLPIPERIDIN-4-YL)-2-(4-PHENOXYPHENYL)-4,5,6,7-TETRAHYDROPYRAZOLO[1,5-A]PYRIMIDINE-3-CARBOXAMIDE OR ITS SALTS THEREOF

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Satyanarayana REVU, Hyderabad, Telangana (IN); Srinivas Reddy GADE, Hyderabad, Telangana (IN); Malla Reddy ADLA, Hyderabad, Telangana (IN); Naveen RAGAM, Hyderabad, Telangana (IN)

(73) Assignee: MSN LABORATORIES PRIVATE LIMITED, R&D CENTER, Hyderabad, Telangana (IN)

(21) Appl. No.: 18/252,882

(22) PCT Filed: Nov. 15, 2022
(86) PCT No.: PCT/IN2021/051072

§ 371 Date: May 14, 2023

(30) Foreign Application Priority Data
Nov. 13, 2020 (IN) 202041049747
Jul. 21, 2021 (IN) 202141032810
Nov. 15, 2021 (WO) PCT/IN2021/051072

(51) Int. Cl. C07D 487/04

(52) U.S. Cl. CPC C07D 487/04

(57) ABSTRACT

The present invention relates to a novel process for the preparation of (S)-7-(1-acryloylpiperidin-4-yl)-2-(4-phenoxyphenyl)-4, 5, 6, 7-tetrahydropyrazolo[1,5-a] pyrimidine-3-carboxamide of formula-1 or its salts. The present invention also relates to novel process for the preparation of intermediate compound of Formula-7 and recovery of the intermediate compound of Formula-12, which is used in the preparation of compound of Formula-7. The compounds of formula-1 & formula-7 are represented by following structural formulae.

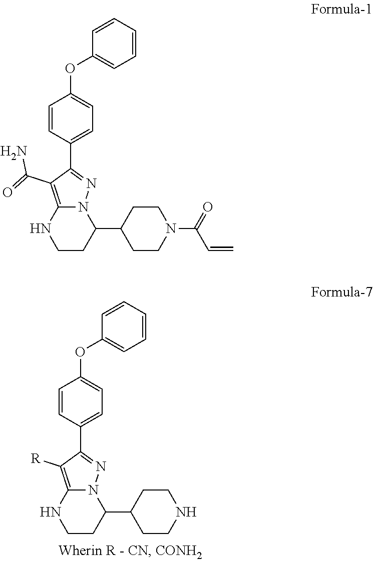

### RELATED APPLICATION

This application claims the benefit of priority to our Indian patent application numbers 202041049747 filed on Nov. 13, 2020 and 202141032810 filed on Jul. 21, 2021, the disclosures of all that are incorporated by reference in their entirety.

### FIELD OF THE INVENTION

The present invention relates to a novel process for the preparation of (S)-7-(1-acryloylpiperidin-4-yl)-2-(4-phenoxyphenyl)-4,5,6,7-tetrahydropyrazolo[1,5-a] pyrimidine-3-carboxamide of formula-1 or its salts. The present invention also relates to process for the preparation of intermediate compound of Formula-7 and recovery of the intermediate compound of Formula-12, which is used in the preparation of compound of Formula-7. The compounds of formula-1 & formula-7 are represented by following structural formulae.

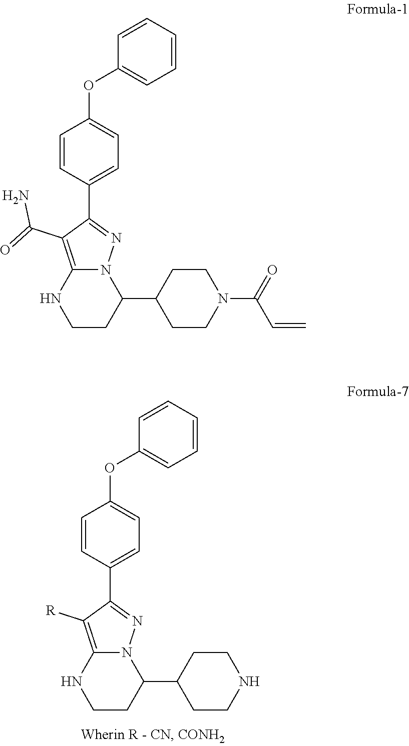

### BACKGROUND OF THE INVENTION

(S)-7-(1-acryloylpiperidin-4-yl)-2-(4-phenoxyphenyl)-4,5,6,7 tetrahydropyrazolo [1,5-a] pyrimidine-3-carboxamide compound of formula-1 is commonly known as Zanubrutinib, which was approved in US under the brand name of Brukinsa for the treatment of adult patients with mantle cell lymphoma (MCL) who have received at least one prior therapy.

Zanubrutinib or pharmaceutically acceptable salts are described in U.S. Pat. No. 9,447,106 B2. This patent also discloses the process for the preparation of Zanubrutinib.

U.S. Ser. No. 10/927,117B2 describes the crystalline form of Zanubrutinib and novel process for the preparation of Zanubrutinib of formula-1.

There is always a need for simple, cost-effective, viable, environment friendly and commercially scalable process for the preparation of Zanubrutinib of Formula-1 and its intermediates thereby results in yield improvement.

### BRIEF DESCRIPTION OF THE INVENTION

The first embodiment of the present invention provides a novel process for preparation of Zanubrutinib of formula-1.

The second embodiment of the present invention provides a novel process for preparation of Zanubrutinib of formula-1.

The third embodiment of the present invention provides a novel compound of formula-5.

The fourth embodiment of the present invention provides a process for preparation of Zanubrutinib of formula-1.

The fifth embodiment of the present invention provides a process for preparation of Zanubrutinib of formula-1.

The sixth embodiment of the present invention provides an amorphous form of Zanubrutinib, which is characterized by powder X-ray diffraction pattern.

The seventh embodiment of the present invention provides a process for recovery of R-isomer compound of Formula-8b and convert it into racemic compound of Formula-7a. The recovery process thereby increases the yield of Zanubrutinib and hence, the wastage of undesired R-isomer is avoided by restoring and racemized to get the compound of formula-7a that is further converted into Zanubrutinib.

The eighth embodiment of the present invention relates to an efficient method for synthesizing intermediate compound of Formula-7a, which is used in the preparation of Zanubrutinib of Formula-1.

The ninth embodiment of the present invention provides a crystalline form of compound of Formula-8a, herein after designated as crystalline form-M characterized by powder X-ray diffractogram.

### BRIEF DESCRIPTION OF THE DRAWINGS

FIG. 1: Illustrates the powder X-Ray diffraction pattern of an amorphous form of compound of formula-1.

FIG. 2: Illustrates the powder X-Ray diffraction pattern of Crystalline Form-M of compound of formula-8a.

### DETAILED DESCRIPTION OF THE INVENTION

The “solvent” used in the present invention is selected from but not limited to “hydrocarbon solvents” such as n-pentane, n-hexane, n-heptane, cyclohexane, petroleum ether, benzene, toluene, xylene and mixtures thereof; “ether solvents” such as dimethyl ether, diethyl ether, diisopropyl ether, methyl tert-butyl ether, 1,2-dimethoxyethane, tetrahydrofuran, 1,4-dioxane and mixtures thereof; “ester solvents” such as methyl acetate, ethyl acetate, n-propyl acetate, isopropyl acetate, n-butyl acetate, isobutyl acetate, tert-butyl acetate and mixtures thereof; “polar-aprotic solvents” such as dim ethyl acetamide, dimethylformamide, dimethylsulfoxide, N-methylpyrrolidone (NMP) and mixtures thereof; “chloro solvents” such as dichloromethane, dichloroethane, chloroform, carbon tetrachloride and mixtures thereof; “ketone solvents” such as acetone, methyl ethyl ketone, methyl isobutyl ketone and mixtures thereof; “nitrile solvents” such as acetonitrile, propionitrile, isobutyronitrile and mixtures thereof; “alcohol solvents” such as methanol, ethanol, n-propanol, iso-propanol, n-butanol, iso-butanol, 2-butanol, tert-butanol, ethane-1,2-diol, propane-1,2-diol and mixtures thereof; “polar solvents” such as water; formic acid, acetic acid and the like or mixture of any of the afore mentioned solvents.

The term “base” used in the present invention refers to inorganic bases selected from “alkali metal carbonates” such as sodium carbonate, potassium carbonate, lithium carbonate, cesium carbonate and the like; “alkali metal bicarbonates” such as sodium bicarbonate, potassium bicarbonate, lithium bicarbonate, cesium bicarbonate and the like; “alkali metal hydroxides” such as sodium hydroxide, potassium hydroxide, lithium hydroxide and the like; “alkyl metals” such as n-butyl lithium and like; “metal hydrides” such as lithium hydride, sodium hydride, potassium hydride and the like; “alkali metal phosphates” such as disodium hydrogen phosphate, dipotassiumhydrogen phosphate; ammonia such as aqueous ammonia, ammonia gas, methanolic ammonia and like and “organic bases” selected from but not limited to methyl amine, ethyl amine, diisopropyl amine, diisopropylethyl amine (DIPEA), diisobutylamine, triethylamine, tert.butyl amine, pyridine, 4-dimethylaminopyridine (DMAP), N-methyl morpholine (NMM), n-methyl pyridine (NMP), 1,8-diazabicyclo[5.4.0. O]undec-7-ene (DBU), 1,5-diazabicyclo[4.3.0]non-5-ene (DBN), 1,4-diazabicyclo[2.2.2]octane (DABCO), imidazole; “alkali metal alkoxides” such as sodium methoxide, sodium ethoxide, potassium methoxide, potassium ethoxide, sodium tert.butoxide, potassium tert.butoxide and the like; “alkali metal amides” such as sodium amide, potassium amide, lithium amide, lithium diisopropyl amide (LDA), sodium bis(trimethylsilyl)amide (NaHMDS), potassium bis(trimethylsilyl)amide, lithium bis(trimethysilyl)amide (LiHMDS) and the like; or mixtures thereof.

As used herein the term “acid” in the present invention refers to inorganic acid and organic acid; inorganic acid is selected from such as hydrochloric acid, hydrobromic acid, sulfuric acid, nitric acid, phosphoric acid, sulfuric acid; organic acids such as acetic acid, maleic acid, malic acid, oxalic acid, succinic acid, fumaric acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (1S)-(+)-camphor sulfonic acid, (1R)-(+)-bromocamphor-10-sulfonic acid, (1S)-(−)-bromocamphor-10-sulfonic acid, (−)-Dibenzoyl-L-tartaric acid (L-DB TA), (−)-Dibenzoyl-L-tartaricacid monohydrate, (+)-Dibenzoyl-D-tartaric acid (D-DBTA), (+)-Dibenzoyl-D-tartaric acid monohydrate, (+)-dipara-tolyl-D-tartaric acid (D-DTTA), (−)-dipara-tolyl-L-tartaricacid (L-DTTA), L(−)-pyroglutamic acid, L(+)-pyroglutamic acid, (−)-lactic acid; or chiral amino acid selected from but not limited to D-isomers and L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, norleucine, norvaline, hydroxyproline, sarcosine, citrulline, cysteic acid, t-butylglycine, t-butylalanine, phenylglycine, cyclohexylalanine, N-acetyl-leucine and the like.

As used herein the term “chiral acid” in the present invention is selected from but not limited to S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (1S)-(+)-camphor sulfonic acid, (1R)-(+)-bromocamphor-10-sulfonic acid, (1S)-(−)-bromocamphor-10-sulfonic acid, (−)-Dibenzoyl-L-tartaric acid (L-DBTA), (−)-Dibenzoyl-L-tartaricacid monohydrate, (+)-Dibenzoyl-D-tartaric acid (D-DBTA), (+)-Dibenzoyl-D-tartaric acid monohydrate, (+)-dipara-tolyl-D-tataric acid (D-DTTA), (−)-dipara-tolyl-L-tartaricacid (L-DTTA), L(−)-pyroglutamic acid, L(+)-pyroglutamic acid, (−)-lactic acid; or chiral amino acid selected from but not limited to D-isomers and L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, norleucine, norvaline, hydroxyproline, sarcosine, citrulline, cysteic acid, t-butylglycine, t-butylalanine, phenylglycine, cyclohexylalanine, N-acetyl-leucine and the like.

The term “salt” used in the present invention refers to acid addition salts selected from inorganic acids such as hydrochloric acid, hydrobromic acid, sulfuric acid, nitric acid, phosphoric acid; organic acids such as acetic acid, maleic acid, malic acid, oxalic acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (1S)-(+)-camphor sulfonic acid (1R)-(+)-bromocamphor-10-sulfonic acid, (1S)-(−)-bromocamphor-10-sulfonic acid, (−)-Dibenzoyl-L-tartaric acid, (−)-Dibenzoyl-L-tartaricacid monohydrate, (+)-Dibenzoyl-D-tartaric acid, (+)-Dibenzoyl-D-tartaric acid monohydrate, (+)-dipara-tolyl-D-tataric acid, (−)-dipara-tolyl-L-tartaricacid, L(−)-pyroglutamic acid, L(+)-pyroglutamic acid, (−)-lactic acid; or chiral amino acid and the like.

As used herein the term “amino protecting group” is selected from but not limited to tert-butyloxycarbonyl (Boc), benzyl, 4-methoxybenzyl, 3,4-dimethoxy benzyl, p-methoxyphenyl, acetyl, propionyl, butyryl, phenylacetyl, toluyl, Phenoxyacetyl, benzoyl, tosyl, methoxycarbonyl, ethoxycarbonyl, 2,2,2-trichloroethoxycarbonyl, 2-iodo ethoxycarbonyl, carbobenzyl (CBZ), 4-methoxybenzyloxycarbonyl, (Fluoren-9-ylmethoxy)carbonyl (Fmoc), 4-m ethoxy-2,3,6-trimethylbenzenesulphonyl (Mtr), benzyl carbamate, acetamide, phthalimide, benzylamine and p-toluenesolfonamide, alkyl trifluoroacetyl such as methyl trifluoroacetyl, ethyl trifluoroacetyl, isopropyl trifluoroacetyl, vinyl trifluoroacetyl and the like.

As used herein the term “deprotection” can be carried out in presence of a suitable “deprotection agent” is selected from but not limited to acids such as hydrochloric acid, hydrobromic acid, hydrofluoric acid, sulfuric acid, nitric acid, phosphoric acid, acetic acid, trifluoroacetic acid, formic acid, substituted/unsubstituted alkyl/aryl sulfonic acids such as methanesulfonic acid, ethanesulfonic acid, benzenesulfonic acid, p-toluenesulfonic acid, pyridinium p-toluene sulfonic acid, trifluromethane sulfonic acid optionally in combination with alcohols and “hydrogen fluoride (HF) sources” such as ammonium fluoride, tetrabutyl ammonium fluoride, pyridine-HF, Et₃N-3HF etc; metal catalysts in presence of hydrogen source and the like;

As used herein the term “reducing agent” used in the present invention is selected from but not limited to Metal hydride including LiAlH₄, NaAlH₄, NaBH₄, KBH₄, mixture of NaBH₄ & acetic acid, mixture of NaBH₄ & trifluoroacetic acid, mixture of NaBH₄ & iodine, mixture of NaBH₄ & trimethylchlorosilane, mixture of NaBH₄ & magnesium chloride, mixture of NaBH₄ & calcium chloride, mixture of NaBH₄ & one of transition metal chlorides, mixture of sodium borohydride BF₃·etherate, sodium cyanoborohydride, sodium triacetoxy borohydride, Aluminium hydride (AlH₃), diisobutylaluminium hydride (DIBAL), Vitride {=Sodium bis(2-methoxyethoxy) aluminum hydride}, Lithium Tri-tert-butoxyaluminum Hydride, Tributyltin Hydride; boranes such as but not limited to BH₃-tetrahydrofuran, BH₃-dimethyl sulfide; hydrazine; metal including but not limited to Na, Fe, Ni, Zn, Sn in presence of acidic medium; Na-liquid ammonia; Silanes including but not limited to tri(C₁-C₆)alkylsilanes, tri(C₁-C₆)alkylsilyl halides; catalytic hydrogenation in presence of transition metals catalysts including but not limited to Ni, Pd, Pt, Rh, Re, Ru and Ir, including their oxides, hydroxides, acetates and combinations thereof, Raney nickel, palladium catalyst such as Pd/C, Pd(OH)₂/C, palladium acetate, Pd/SrCO₃, Pd/Al₂O₃, Pd/MgO, Pd/CaCO₃, Pd/BaSO₄, PdO, PdCl₂, Rh/C, Ru/C, Re/C, Pt/C, platinum oxide, platinum black, PtO₂, Rh/C, RuO₂ and the like.

The term “oxidizing agent” used in the present invention is selected from but not limited to sodium hypochlorite in presence of a catalyst like TEMPO (2,2,6,6-tetramethyl-1-piperidinyloxy, free radical)/KBr; hydrogen peroxide, NBS-benzoyl peroxide, cumene hydro peroxide, peracids such as peracetic acid, trifluoro peracetic acid, perbenzoic acid, m-chloroperbenzoic acid (MCPBA); 4,5-dichloro-3,6-dihydroxyphthalonitrile (DDQ), 2,3,5,6-tetrachlorocyclohexa-2,5-diene-1,4-dione (Chloranil), potassium permanganate (KMnO4), Manganese dioxide (MnO₂) and the like.

The process for the preparation of Zanubrutinib of present application is schematically represented below:

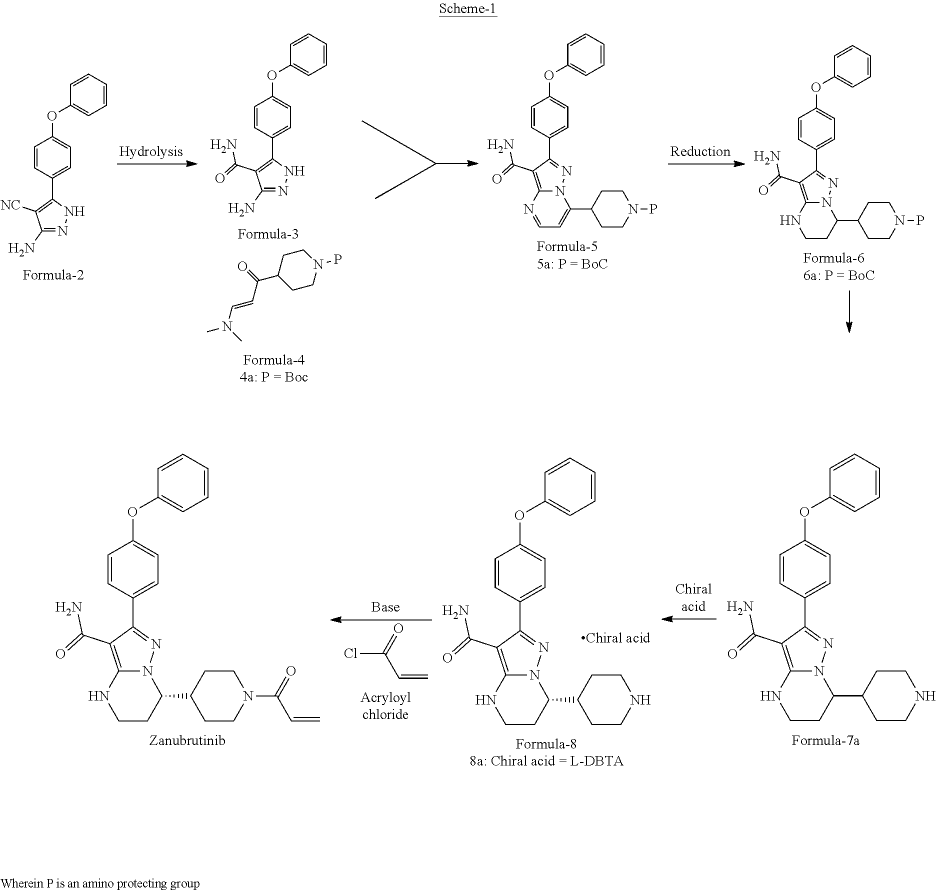

The first embodiment of present invention provides a novel process for the preparation of Zanubrutinib of formula-1 or a salt thereof comprising,
a) hydrolysis of compound of formula-2 to provide compound of formula-3 or a salt thereof,
b) reacting compound of formula-3 with compound of formula-4 to provide compound of formula-5,
c) converting compound of formula-5 into Zanubrutinib of formula-1 or a salt thereof.

In first aspect of first embodiment of the present invention, hydrolysis of compound of formula-2 can be carried out in presence of an acid or a base as defined above.

In second aspect of first embodiment of the present invention, hydrolysis of compound of formula-2 can be carried out in a solvent that is selected from alcohol solvents, chloro solvents, ether solvents, polar aprotic solvents, polar solvents, ester solvents, ketone solvents, nitrile solvents, hydrocarbon solvents and/or mixtures thereof.

In third aspect of first embodiment of the present invention, reaction between compound of formula-3 and compound of formula-4 can be carried out in presence of an acid as defined above.

In fourth aspect of the first embodiment of the present invention, reaction between compound of formula-3 and compound of formula-4 can be carried out in a solvent that is selected from alcohol solvents, chloro solvents, ether solvents, ester solvents, ketone solvents, nitrile solvents, hydrocarbon solvents, polar aprotic solvents, polar solvents and/or mixtures thereof.

The second embodiment of present invention provides a novel process for the preparation of Zanubrutinib of formula-1 or a salt thereof comprising,
a) reduction of compound of formula-5 to provide compound of formula-6,
b) deprotecting the compound of formula-6 to provide the compound of formula-7a or salts thereof,
c) converting the compound of formula-7a or salts thereof into Zanubrutinib of formula-1 or a salt thereof.

In first aspect of second embodiment of the present invention, reduction of compound of formula-5 can be carried out in presence of reducing agent as defined above.

In second aspect of second embodiment of the present invention, reduction of compound of formula-5 can be carried out in a solvent that is selected from alcohol solvents, chloro solvents, ether solvents, ester solvents, ketone solvents, nitrile solvents, hydrocarbon solvents, polar aprotic solvents, polar solvents and/or mixtures thereof.

In third aspect of second embodiment of the present invention, deprotection in step-b) is carried out in presence of a deprotecting agent as defined above.

In fourth aspect of second embodiment of the present invention, converting compound of formula-7a or salts thereof to Zanubrutinib of formula-1 in step-b) can be done by processes as disclosed in the literature U.S. Pat. No. 9,447,106 B2 or other available references or the present application.

The third embodiment of present invention provides a novel compound of formula-5.

In an aspect of the third embodiment, the novel compound of formula-5 is useful in the preparation of Zanubrutinib of formula-1.

The process for the preparation of Zanubrutinib of present application is schematically represented below:

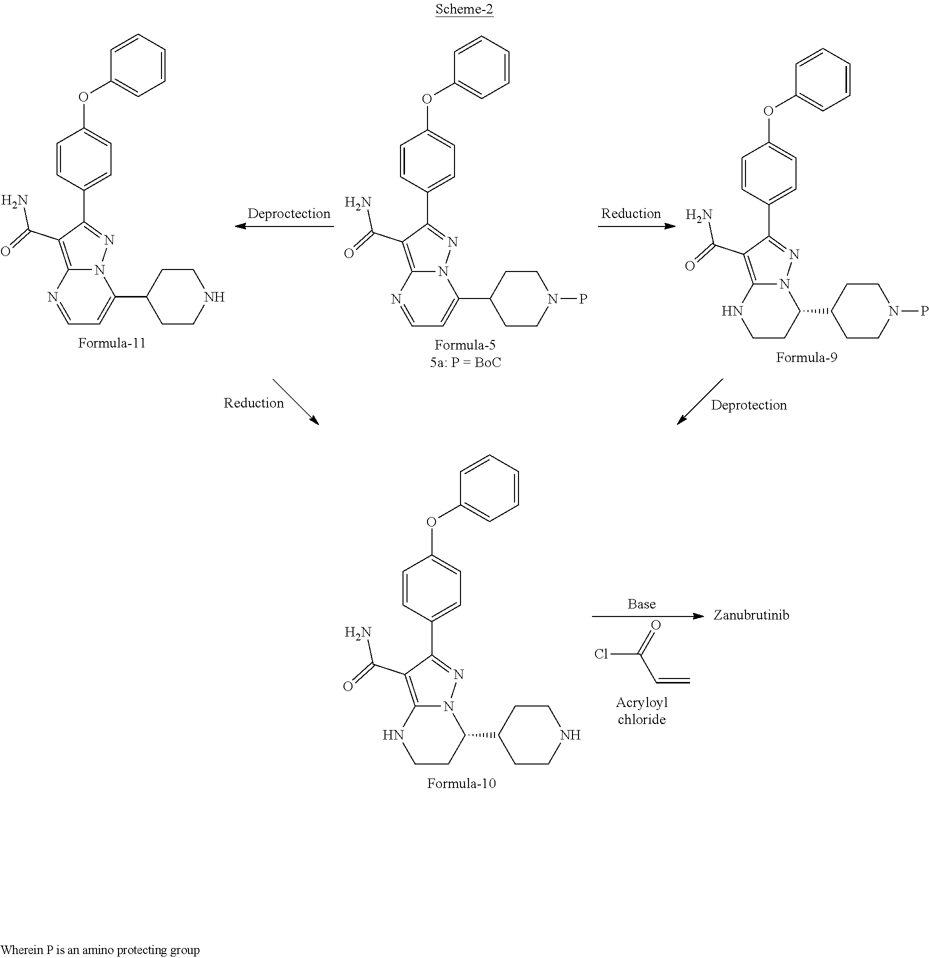

The fourth embodiment of the present invention provides a process for the preparation of Zanubrutinib of formula-1 or a salt thereof comprising,
a) reduction of compound of formula-5 in presence of reducing agent in a solvent to provide compound of formula-9 or a salt thereof,
b) deprotection of compound of formula-9 with a deprotecting agent in a solvent to provide compound of formula-10 or a salt thereof,
c) reacting compound of formula-10 with acryloyl chloride in presence of a base in a solvent to provide Zanubrutinib compound of formula-1 or a salt thereof.

Suitable reducing agent in step-a) is selected from β-chlorodiisopinocampheyl borane (DIP Chloride); or a reducing agent like, NaBH4, borane THF or borane DMS in combination with a chiral catalyst like (R)-tetrahydro-1-methyl-3,3-diphenyl-1H,3H-pyrrolo(1,2-c)(1,3,2)oxazaborole; chiral acids; iridium catalyst system including, but not limited to, [Ir(COD)Cl]₂/(R or S)-MeO-Biphep, [Ir(COD)Cl]₂/(R or S)-Binap, [Ir(COD)Cl]₂/(R or S)-Tol-Binap, [Ir(COD)Cl]₂/(R or S)-xyl-Binap, [Ir(COD)Cl]₂/(S,S or R,R)-Diop, [Ir(COD)Cl]₂/(R or S)—P-Phos, [Ir(COD)Cl]₂/(R or S)-Tol-P-Phos, [Ir(COD)Cl]₂/(R or S)-Xyl-P-Phos, [Ir(COD)Cl]₂/(R,R or S,S)-Me-DuPhos, [Ir(COD)Cl]₂/(R or S)-SegPhos, [Ir(μ-Cl)(cod)]₂/(R or S)-Ship, [Ir(μ-Cl)(cod)]₂/(R or S)-Siphos, [Ir(μ-Cl)(cod)]₂/(R or S)-Siphos-PE, [Ir(μ-Cl)(cod)]₂/(R or S)-MonoPhos, [Ir(μ-Cl)(cod)]₂/(R or S)-tol-SDP, [Ir(μ-Cl)(cod)]₂/(S,S or R,R)-Diop, [Ir(μ-Cl)(cod)]₂/(S,R or R,S)-Josiphos, [Ir(μ-Cl)(cod)]₂/(R or S)-Binap, [Ir(μ-Cl)(cod)]₂/(R or S)-MeO-Biphep, [Ir(μ-Cl)(cod)]₂/(R or S)-Synphos, or [Ir(μ-Cl)(cod)]₂/(R or S)-Difluorphosor [Ir(cod)₂]+X— (X: e.g. BF₄, NO₃, OTf, PF₆, SbF₆ and BarF) and ligands; a rhodium catalyst system including, but not limited to, [Rh(COD)₂]BF₄ and ligands; or, a ruthenium catalyst system including, but not limited to, RuCl₂ (R or S)-Binap/(R or S)-Daipen, RuCl₂ (R or S)-Binap/(R,R or S,S)-Dpen, RuCl₂(S or R)-Binap (S,S or R,R)-Dach, RuCl₂[(R or S)-Tol-Binap][(S,S or R,R)-Dpen], RuCl₂ (R,R or S,S)-Me-Duphos/(R,R or S,S)-Dpen, RuCl₂ (R,R or S,S)-Et-DuPhos/(R,R or S,S)-Dpen, RuCl₂ (R,R or S,S)-Et-DuPhos/(R,R or S,S)-Dach, RuCl₂(S,S or R,R)-i-Pr-DuPhos/(R,R or S,S)-Dpen, RuCl₂ (R or S)-HexaPhemp/(R,R or S,S)-Dpen, RuCl₂ (R or S)-MeO-Biphep/(R,R or S,S)-Dpen.

Suitable deprotecting agent in step-b) is selected from “deprotecting agent” as defined above; suitable base in step-c) is selected from organic or inorganic base as defined above and suitable solvent in step-a) to step-c) is selected from “solvent” as defined above.

The fifth embodiment of the present invention provides a process for the preparation of Zanubrutinib of formula-1 or a salt thereof comprising,
a) deprotection of compound of formula-5 with a deprotecting agent in a solvent to provide compound of formula-11 or a salt thereof,
b) reduction of compound of formula-11 in presence of a reducing agent in a solvent to provide compound of formula-10 or a salt thereof,
c) reacting compound of formula-10 with acryloyl chloride in presence of a base in a solvent to provide Zanubrutinib compound of formula-1 or a salt thereof.

Suitable deprotecting agent in step-a) is selected from “deprotecting agent” as defined above; suitable base in step-c) is selected from organic or inorganic base as defined above; suitable solvent in step-a) to step-c) is selected from “solvent” as defined above; suitable reducing agent in step-b) as defined in second embodiment.

The sixth embodiment of the present invention provides an amorphous form of Zanubrutinib be characterized by PXRD (Powder X-Ray Diffraction) pattern substantially in accordance with figure-1.

The processes for the preparation of racemic compound of Formula-7 and Zanubrutinib of present application are schematically represented below.

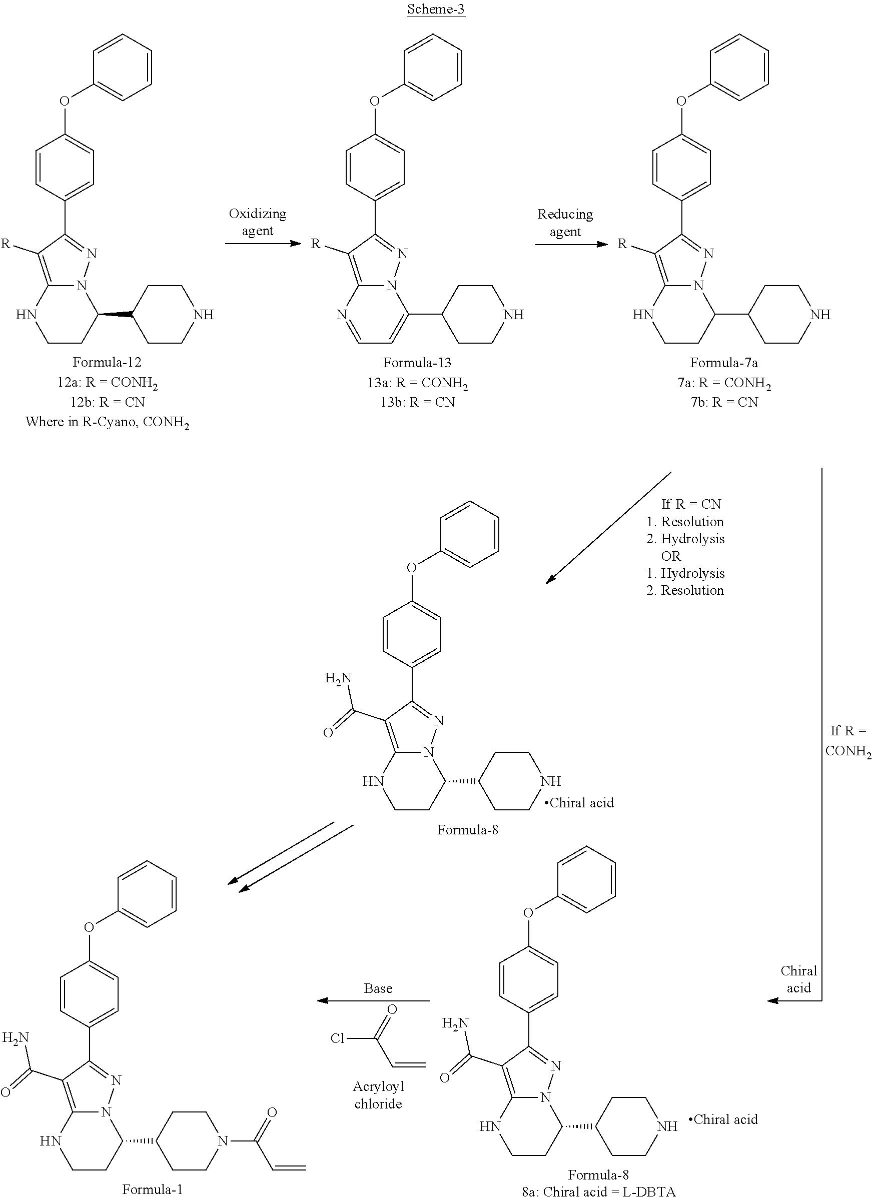

The seventh embodiment of the present invention is to provide a process for the preparation of racemic compound of Formula-7, which comprises:
a) reacting an enantiomerically enriched R-isomer of formula-12 with an oxidizing agent in a solvent to provide a compound of Formula-13;
b) reducing compound of Formula-13 in presence of a reducing agent in a solvent to provide the racemic compound of Formula-7.

In first aspect of seventh embodiment of the present invention, the oxidizing agent used in step-a) is selected from oxidizing agent as described above.

In second aspect of seventh embodiment of the present invention, the solvents used in step—a) & b) are selected from but not limited to hydrocarbon solvents, ether solvents, ester solvents, non-polar aprotic solvents, polar-aprotic solvents, chloro solvents, ketone solvents, nitrile solvents, alcohol solvents, polar solvents such as water, formic acid, acetic acid or their mixture as defined above.

In third aspect of seventh embodiment of the present invention, reducing agent used in step-b) is same as defined above.

The fourth aspect of seventh embodiment of the present invention provides a recovery process for the preparation of an enantiomerically enriched R-isomer of Formula-12, wherein the recovery process of formula-12 comprises:
a) distilling the solvent from the filtrate obtained in the resolution of racemic compound of formula-7 diasteromeric salt which contains enantiomerically enriched R-isomer of Formula-12 with a chiral acid;
b) treating the obtained compound in step-a) with a base;
c) isolating the enantiomerically enriched R-isomer compound of Formula-12.

In fifth aspect of seventh embodiment of the present invention, the base used in step-b) is selected from organic or inorganic bases as defined above.

In sixth aspect of seventh embodiment of the present invention, Formula-12 used in the process of the present invention is either free of S-isomer or is enantiomerically enriched. For an instance, Formula-12 has a chiral purity of at least about 0.5%, 5%, 10%, 20%, 30%, 40%, 50%, 60%, 70%, 80%, 90%, 95%, 97%, 98%, 99% or more; for example, about 99.5% or about 99.9% ee (enantiomeric excess) as measured by a chiral HPLC method.

In an eighth embodiment of the present invention, Formula-7 is Formula-7a in step-a) wherein the filtrate is obtained from resolution of racemic compound of formula-7a according to the procedure disclosed in the present application. The structure of compound of Formula-7a is as depicted below:

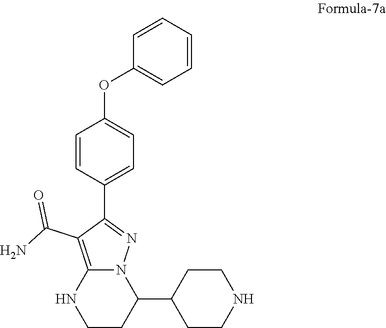 [SMILES: NC(=O)c1c(-c2ccc(Oc3ccccc3)cc2)nn2c1NCCC2C1CCNCC1]

Alternatively, Formula-7 is Formula-7b in step-a) wherein the filtrate is obtained from resolution of racemic compound of formula-7b according to the procedure disclosed in Example-1 of the patent document U.S. Ser. No. 10/927,117B2 herein included by reference. The structure of compound of Formula-7b is as depicted below:

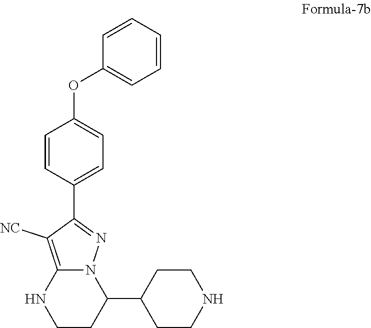 [SMILES: N#Cc1c(-c2ccc(Oc3ccccc3)cc2)nn2c1NCCC2C1CCNCC1]

The ninth embodiment of the present invention provides a crystalline form of the compound of formula-8a, herein after the said crystalline form is designated as crystalline form-M.

In an aspect of ninth embodiment provides the crystalline form-M of formula-8a characterized by its powder X-Ray diffractogram as illustrated in figure-2.

In tenth embodiment of the present invention, the compound of Formula-7a, the compound of Formula-7b and crystalline form-M of formula-8a which are obtained according to the present application are further converted into Zanubrutinib of Formula-1 by the process described in prior art references such as U.S. Pat. No. 9,447,106B2, U.S. Ser. No. 10/927,117B2 and/or as described in the present application.

The intermediate compounds of Zanubrutinib according to the present invention having chemical purity greater than about 98%, preferably greater than about 99%, more preferably greater than about 99.5% by HPLC (High Performance Liquid Chromatography).

Zanubrutinib of formula-1 obtained according to the present invention having chemical purity greater than about 98%, preferably greater than about 99%, more preferably greater than about 99.5% by HPLC (High Performance Liquid Chromatography).

HPLC method of analysis for the compound of formula-1 or its pharmaceutically acceptable salts of the present invention can be carried out by known techniques or by the method disclosed in this application.

### Method of Analysis:

Compound of formula-1 produced by the present invention was analyzed by HPLC under the following conditions;
Apparatus: A liquid chromatograph is equipped with variable wavelength UV detector;
Column: Zorbax SB C8, 250×4.6 mm, 5 μm;
Column temperature: 35° C.;
Wave length: 210 nm;
Injection volume: 10 μL;
Elution: Gradient;
Diluent: Acetonitrile:Water (1:1 v/v);
Buffer: Accurately transfer 1000 ml of milli-Q-water into a suitable cleaned and dry beaker. Transfer 1.0 ml pf Orthophosphoric acid (85%) o above 1000 ml of milli-Q-water, mix well (pH ˜1.95). Filter the above solution through 0.22 μm PVDF membrane filter paper and sonicate to degas it.
Mobile phase-A: Buffer (100%)
Mobile phase-B: Accurately transfer 900 ml of Acetonitrile and 100 ml of milli-Q-water into a 1000 ml Mobile phase bottle and mix well.

Zanubrutinib of formula-1 or a salt thereof prepared according to the present invention can be further micronized or milled in conventional techniques to get the desired particle size to achieve desired solubility profile based on different forms of pharmaceutical composition requirements. Techniques that may be used for particle size reduction include, but not limited to ball milling, roll milling and hammer milling, and jet milling. Milling or micronization may be performed before drying or after the completion of drying of the product.

Zanubrutinib of Formula-1 or a salt thereof prepared according to the present invention having the particle size less than 500 μm, 400 μm, 300 μm, 200 μm or 150 μm.

The eleventh embodiment of the present invention provides pharmaceutical composition comprising Zanubrutinib of formula-1 or a salt thereof and one or more pharmaceutically acceptable excipients.

Wherein, suitable pharmaceutically acceptable excipients selected from but not limited to binders, diluents, disintegrants, surfactants and lubricants. Suitable binders that can be include polyvinylpyrolidone, copovidone, starches such as pregelatinized starch, cellulose derivatives such as hydroxypropylmethyl cellulose, ethylcellulose, hydroxypropylcellulose and carboxymethylcellulose, gelatine, acacia, agar, alginic acid, carbomer, chitosan, dextrates, cyclodextrin, dextrin, glycerol dibehenate, guargum, hypromellose, maltodextrin, poloxamer, polycarbophil, polydextrose, polyethylene oxide, polymethacrylates, sodium alginate, sucrose, mixtures thereof; suitable diluents that can be include anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol starch, calcium lactate or mixtures thereof; suitable disintegrants that can be include magnesium aluminometa silicate (or magnesium aluminum silicate), starch, pregelatinized starch, sodium starch glycolate, crospovidone, croscarmellose sodium, low-substituted hydroxypropyl cellulose, alginic acid, carboxy methyl cellulose sodium, sodium alginate, calcium alginate and chitosan; suitable lubricants that can be include (but are not limited to) magnesium stearate, stearic acid, palmitic acid, talc, and aerosil. Suitable surfactants that can be include (but are not limited to) polysorbate 80, polyoxyethylene sorbitan, polyoxyethylene-polyoxy-propylene copolymer and sodium lauryl sulphate; beta-cyclodextrin include (but are not limited to) sulfobutylalkyl ether-beta-cyclodextrin, betadex-sulfobutyl ether sodium, or hydroxypropyl-beta-cyclodextrin.

As used herein, the term “pharmaceutical composition” or “pharmaceutical formulation” is formulated in a manner suitable for the route of administration to be used which include but are not limited to tablets, pills, powders, liquids, suspensions, emulsions, granules, capsules, suppositories, or injection preparations.

In twelfth embodiment of the present invention, a pharmaceutical composition comprising Zanubrutinib of formula-1 or a salt thereof prepared according to the present invention and one or more pharmaceutically acceptable excipients used for the treatment of adult patients with mantle cell lymphoma (MCL) who have received at least one prior therapy.

The best mode of carrying out the present invention is illustrated by the below mentioned examples. These examples are provided as illustration only and hence should not be considered as limitation of the scope of the invention.

### EXAMPLES

### Example-1: Preparation of (E)-Tert-butyl 4-(3-(dimethylamino)acryloyl)piperidine-1-carboxylate of formula-4a

A mixture of Tert-butyl 4-acetyl piperidine-1-carboxylate (25 g), Dimethylformamide-dimethyl acetal (62.5 ml) and dimethylformamide (62.5 ml) was heated to 110-115° C. and stirred. Distilled off the solvent from the reaction mixture. Dichloromethane and aqueous ammonium chloride solution were added to the obtained compound at 25-30° C. Separated the organic layer from the mixture. Distilled off the solvent from the organic layer and co-distilled with n-hexane. n-Hexane was added to the obtained compound and cooled to 0-5° C. Filtered the solid, washed with n-hexane and dried to get the title compound.

Yield: 25.5 g.

### Example 2: Preparation of (E)-Tert-butyl 4-(3-(dimethylamino)acryloyl)piperidine-1-carboxylate of formula-4a

A mixture of Tert-butyl 4-acetyl piperidine-1-carboxylate (100 g), Dimethylformamide-dimethyl acetal (400 ml) and dimethylformamide (250 ml) were heated to 110-115° C. and stirred. Distilled off the solvent from the reaction mixture. Dichloromethane and water were added to the obtained residue at 25-30° C., separated the organic layer and washed with aqueous ammonium chloride solution. Distilled off the solvent and co-distilled with n-heptane. The obtained solid was slurried in n-Heptane and dried to get the title compound.

Yield: 108.4 g.

### Example 3: Preparation of 3-amino-5-(4-phenoxyphenyl)-1H-pyrazole-4-carboxamide of formula-3

A mixture of methane sulfonic acid (80 ml) and 5-Amino-3-(4-phenoxyphenyl)-1H-pyrazole-4-carbonitrile (20 g) were heated to 85-90° C. and stirred. Cooled the reaction mixture to 25-30° C. and stirred. Aqueous sodium hydroxide solution was added to the resultant mixture and stirred. Filtered the solid, washed with water and dried to get the title compound.

Yield: 17.2 g.

### Example 4: Preparation of 3-amino-5-(4-phenoxyphenyl)-1H-pyrazole-4-carboxamide of formula-3

A mixture of methane sulfonic acid (100 ml), water (2.5 ml) and 5-Amino-3-(4-phenoxyphenyl)-1H-pyrazole-4-carbonitrile (25 g) were heated to 65-70° C. and stirred. Cooled the reaction mixture to 10-15° C., water was added and stirred at the same temperature. Aqueous sodium hydroxide solution was added to the resultant mixture and stirred at 30-35° C. Filtered the solid, washed with water. The obtained compound was slurried in water followed by in the mixture of water and methanol and dried to get the title compound.

Yield: 23.2 g.

### Example 5: Preparation of Tert-butyl 4-(3-carbamoyl-2-(4-phenoxyphenyl) pyrazolo[1,5-a]pyrimidin-7-yl)piperidine-1-carboxylate of formula-5a

A mixture of 3-amino-5-(4-phenoxyphenyl)-1H-pyrazole-4-carboxamide of formula-3 (17 g), (E)-Tert-butyl 4-(3-(dimethylamino)acryloyl)piperidine-1-carboxylate of formula-4a (19.6 g), toluene (136 ml) and acetic acid (8.5 ml) were heated to 105-110° C. Stirred the reaction mixture at same temperature and collected water by azeotropically. Distilled off the solvent completely from the reaction mixture and co-distilled with methanol. Slurried the obtained compound in methanol and dried to get the title compound.

Yield: 26 g. M.P: 230° C.

### Example 6: Preparation of Tert-butyl 4-(3-carbamoyl-2-(4-phenoxyphenyl) pyrazolo[1,5-a]pyrimidin-7-yl)piperidine-1-carboxylate of formula-5a

A mixture of 3-amino-5-(4-phenoxyphenyl)-1H-pyrazole-4-carboxamide of formula-3 (50 g), (E)-Tert-butyl 4-(3-(dimethylamino)acryloyl)piperidine-1-carboxylate of formula-4a (50.4 g) and acetic acid (250 ml) were heated to 45-50° C., stirred the reaction mixture at same temperature. The reaction mixture was cooled to 30-35° C. and water was added slowly to obtain the solid. The obtained solid was filtered, washed with water and the obtained compound was slurried in water and dried to get the title compound.

Yield: 78 g.

### Example 7: Preparation of Tert-butyl 4-(3-carbamoyl-2-(4-phenoxyphenyl)-4,5,6,7 tetrahydro pyrazolo[1,5-a]pyrimidin-7-yl)piperidine-1-carboxylate of formula-6a

Sodium borohydride (5.4 g) was added in lot-wise to the solution of tert-butyl 4-(3-carbamoyl-2-(4-phenoxyphenyl)pyrazolo[1,5-a]pyrimidin-7-yl)piperidine-1-carboxylate of formula-5a (25 g) in ethanol (500 ml) at 25-30° C. Heated the reaction mixture to 60- and stirred the reaction mixture at same temperature. Distilled off the solvent from the reaction mixture. Dichloromethane was added to the obtained compound at 25-30° C. then washed the mixture with aqueous sodium chloride solution. Distilled off the solvent from the dichloromethane layer and co-distilled with n-heptane. Slurried the obtained compound in n-heptane and dried to get the title compound.

Yield: 22 g.

### Example 8: Preparation of Tert-butyl 4-(3-carbamoyl-2-(4-phenoxyphenyl)-4,5,6,7 tetrahydro pyrazolo[1,5-a]pyrimidin-7-yl)piperidine-1-carboxylate of formula-6a

To the mixture of methanol (300 ml), acetic acid (150 ml) and tert-butyl 4-(3-carbamoyl-2-(4-phenoxyphenyl)pyrazolo[1,5-a]pyrimidin-7-yl)piperidine-1-carboxylate of formula-(30 g), 5% Pd/C (3 g) was charged into autoclave at 30-35° C. 4-5 Kg/cm 2 of Hydrogen pressure was applied to the reaction mixture, heated to 45-50° C. and stirred at the same temperature. The reaction mixture was cooled to 30-35° C. and dichloromethane was added. Filtered the reaction mixture through hyflo bed and washed with dichloromethane. The filtrate was distilled and co-distilled with methanol. To the obtained compound, methanol was added and heated to 55-60° C. and stirred at the same temperature. Cooled to 30-35° C., water was added slowly and stirred. Filtered the solid, washed with water.

The above-obtained compound was slurried in methanol and dried to get the title compound.

Yield: 24.4 g.

### Example 9: Preparation of 2-(4-Phenoxyphenyl)-7-(piperidin-4-yl)-4,5,6,7-tetrahydro pyrazolo pyrimidine-3-carbonitrile of formula-7a

Isopropanolic HCl (60 ml) was added to the mixture of tert-butyl 4-(3-carbamoyl-2-(4-phenoxyphenyl)-4,5,6,7 tetrahydropyrazolo[1,5-a]pyrimidin-7-yl)piperidine-1-carboxylate of formula-6a (20 g) and dichloromethane (120 ml) at 25-30° C. and stirred the reaction mixture at same temperature. Cooled the reaction mixture to 0-5° C., methyl tert-butyl ether (80 ml) was added to it and stirred. Filtered the obtained solid and washed with n-hexane. Slurried the compound in n-hexane. Water and aqueous sodium hydroxide solution were added to the obtained compound and stirred. Filtered the solid, washed with water and dried to get the title compound.

Yield: 14 g.

### Example 10: Preparation of 2-(4-Phenoxyphenyl)-7-(piperidin-4-yl)-4,5,6,7-tetrahydro pyrazolo pyrimidine-3-carbonitrile of formula-7a

To the mixture of water (39 ml) and tert-butyl 4-(3-carbamoyl-2-(4-phenoxyphenyl)-4,5,6,7 tetrahydropyrazolo[1,5-a]pyrimidin-7-yl)piperidine-1-carboxylate of formula-6a (78 g), dilute Hydrochloric acid (39 ml) was added at 30-35° C. and stirred at the same temperature. To the reaction mass, dichloromethane was added and both the aqueous and organic layers were separated. The aqueous layer was cooled to 10-15° C. and dichloromethane was added to it. Aqueous sodium hydroxide was charged to the reaction mixture and temperature raised to 30-35° C. Separated the organic layer and extracted the aqueous layer with dichloromethane, combined the organic layers and washed with water. Distilled out the organic layer and co-distilled with methyl tert-butyl ether followed by n-heptane. To the obtained compound, n-heptane was charged at 30-35° C. and stirred. Filtered the solid and washed with n-heptane, dried to get the title compound.

Yield: 63 g.

### Example 11: Preparation of (S)-2-(4-phenoxyphenyl)-7-(piperidin-4-yl)-4,5,6,7-tetrahydropyrazolo pyrimidine-3-carboxamide. Dibenzoyl-L-tartaric acid compound of formula-8a

Dibenzoyl-L-tartaric acid was added to the solution of 2-(4-Phenoxyphenyl)-7-(piperidin-4-yl)-4,5,6,7-tetrahydro pyrazolo[1,5-a]pyrimidine-3-carbonitrile of formula-7a (7 g) in methanol (52.5 ml) at 25-30° C. Heated the mixture to 50-55° C. and stirred for 2 hours at same temperature. Cooled the mixture to 25-30° C. and stirred. Filtered the solid and washed with methanol. Methanol and water were added to the obtained compound at 25-Heated the mixture to 50-55° C. and stirred. Cooled the reaction mixture to 25-30° C. and stirred. Filtered the solid, washed with methanol and dried to get the title compound.

Yield: 2.5 g.

### Example 12: Preparation of (S)-2-(4-phenoxyphenyl)-7-(piperidin-4-yl)-4,5,6,7-tetrahydropyrazolo [1,5-a]pyrimidine-3-carboxamide. Dibenzoyl-L-tartaric acid compound of formula-8a

Dibenzoyl-L-tartaric acid (9.37 g) was added to the mixture of 2-(4-Phenoxyphenyl)-7-(piperidin-4-yl)-4,5,6,7-tetrahydro pyrazolo[1,5-a]pyrimidine-3-carbonitrile of formula-7a (20 g) and methanol (400 ml) at 30-35° C. Heated the mixture to 55-60° C. and stirred for 2.5 hours at the same temperature. Cooled the mixture to 30-35° C. and stirred. Filtered the solid and washed with chilled methanol. The obtained compound was slurried in the mixture of methanol and water. Converted the obtained salt into its freebase using sodium hydroxide. Repeated the same process one more time to get freebase. Dissolved the free base in the mixture of methanol and water at 50-55° C. The solution of dibenzoyl-L-tartaric acid (2.1 g) in water (9 ml) and methanol (27 ml) was added to it at 50-55° C. and stirred. Cooled the mixture to 25-30° C. and stirred. Filtered the solid, washed with methanol and dried to get pure S-isomer of title compound.

Yield: 3.8 g.

### Example 13: Preparation of (S)-2-(4-phenoxyphenyl)-7-(piperidin-4-yl)-4,5,6,7-tetrahydropyrazolo[1,5-a] pyrimidine-3-carboxamide. Dibenzoyl-L-tartaric acid of formula-8a

The mixture of 2-(4-Phenoxyphenyl)-7-(piperidin-4-yl)-4,5,6,7-tetrahydro pyrazolo[1,5-a] pyrimidine-3-carboxamide of formula-7a (50 g), methanol (675 ml) and water (225 ml) was heated to 50-55° C. The solution of dibenzoyl-L-tartaric acid (23.6 g) in water (25 ml) and methanol (75 ml) was added to the above-obtained solution at 50-55° C. and stirred at same temperature. Cooled the mixture to 25-30° C. and stirred. Filtered the solid and washed with methanol. Converted the obtained salt into its free base using sodium hydroxide. Repeated the same process one more time to get freebase. Dissolved the free base in the mixture of methanol and water at 50-55° C. The solution of dibenzoyl-L-tartaric acid (23.6 g) in water (25 ml) and methanol (75 ml) was added to it at 50-55° C. and stirred. Cooled the mixture to 25-30° C. and stirred. Filtered the solid, washed with methanol and dried to get pure S-isomer of title compound.

Yield: 20 g; PXRD of the obtained compound is depicted in FIG. 2.

### Example 14: Preparation of Zanubrutinib

Sodium bicarbonate (0.9 g) was added to the mixture compound of formula-8a (2 g), acetonitrile (24 ml) and water (25 ml) at 25-30° C. Cooled the reaction mixture to −5° C. to and acryloyl chloride (0.3 g) was added to the reaction mixture at same temperature. Stirred the reaction mixture for 2 hours at same temperature. Ethyl acetate was added to reaction mixture at −5 to 0° C. Separated the layers. Aqueous layer extracted with ethyl acetate and combined organic layers. Washed the organic layer with aqueous sodium bicarbonate solution and aqueous sodium chloride solution. Distilled off the solvent from the organic layer and co-distilled with n-hexane to get the title compound.

Yield: 0.74 g.

### Example 15: Preparation of Zanubrutinib

Aqueous sodium hydroxide solution was added to the mixture of compound of formula-8a (150 g), dichloromethane (1500 ml) and water (1500 ml) at 30-35° C. and stirred. The layers were separated and aqueous layer was extracted with dichloromethane. Organic layers are combined, washed with water and distilled out the solvent under vacuum. Dichloromethane (3120 ml) and diisopropylethyl amine (67.58 g) were added to the obtained compound at 30-35° C. and cooled to −40 to −45° C. Acryloyl chloride (14 ml) was added into the reaction mass and stirred at the same temperature. To the reaction mass, water was added. The temperature of the reaction mass was raised to 0 to 10° C. and layers were separated. The aqueous layer was extracted with dichloromethane. Organic layers were combined together and washed with aqueous sulfuric acid followed by aqueous sodium bicarbonate solution, water and sodium chloride solution. The organic layer was treated with activated carbon at 30-35° C. and distilled out the solvent completely from the filtrate. Dissolved the obtained compound in ethyl acetate at 55-60° C. and treated with activated carbon, distilled. Ethyl acetate was charged into it and stirred at 30-35° C., filtered the solid, washed with ethyl acetate and dried to get the title compound.

Yield: 55.4 g.

### Example 16: Preparation of Amorphous Form of Zanubrutinib

Dissolved the Zanubrutinib (330 g) in methanol (3300 ml) at 30-35° C. and the obtained solution was treated with activated carbon. Distilled off the solvent completely from the obtained filtrate and dried to get the title compound.

Yield: 310 g.

### Example 17: Preparation of Enantiomerically enriched (R)-2-(4-phenoxyphenyl)-7-(piperidin-4-yl)-4,5,6,7-tetrahydropyrazolo[1,5-a] pyrimidine-3-carboxamide of formula-12a

Distilled off the solvent from the filtrate obtained in Example-12 or 13. Dichloromethane and water were added to the obtained compound at 25-30° C. Basified the mixture using aqueous sodium hydroxide solution. Organic layer was separated and the aqueous layer was extracted with dichloromethane. Combined the organic layers and washed with water. Distilled off the solvent from the organic layer to get the title compound.

### Example 18: Preparation of 2-(4-phenoxyphenyl)-7-(piperidin-4-yl)pyrazolo[1,5-a]pyrimidine-3-carboxamide of Formula 13a

Manganese dioxide (5.2 g) is added to the mixture of compound of Formula-12a (5 g) and 1, 4-dioxane (500 ml) at 25-30° C. Heated the reaction mixture to 100-105° C. and stirred. Another lot of Manganese dioxide (5.2 g) is added to the reaction mixture at 100-105° C. and stirred. The reaction mixture is cooled to 25-30° C. and stirred. The reaction mixture is filtered through hyflo and washed with the mixture of 1,4-dioxane and dichloromethane. Water and dichloromethane are added to the obtained filtrate. The organic layer is separated, washed with water and distilled off the solvent under reduced pressure to get the title compound.

Yield: 3.2 g.

### Example 19: Preparation of 2-(4-Phenoxyphenyl)-7-(piperidin-4-yl)-4,5,6,7-tetrahydro pyrazolo pyrimidine-3-carboxamide of Formula 7a

The compound of Formula-12a (3.0 g), acetic acid (15 ml) and methanol (90 ml) are charged into autoclave vessel. Pd/C is added into the above mixture and purged with nitrogen. Hydrogen pressure is applied at 45° C. and stirred. Further heated the reaction mixture to 50-70° C. and stirred. Filtered the mixture through hyflo bed and washed with dichloromethane. Distilled off the solvent and co-distilled with n-heptane to get the residue. The crude compound is purified via column chromatography using methanol and dichloromethane to get the title compound.

Yield: 1 g.

### Example 20-Preparation of Amorphous Form of Zanubrutinib

Dissolved Zanubrutinib (17 g) in methanol (170 ml) at 25-30° C. The obtained solution is filtered and distilled off the solvent from the filtrate to obtain the solid. The obtained solid is dried to get an amorphous form of Zanubrutinib.

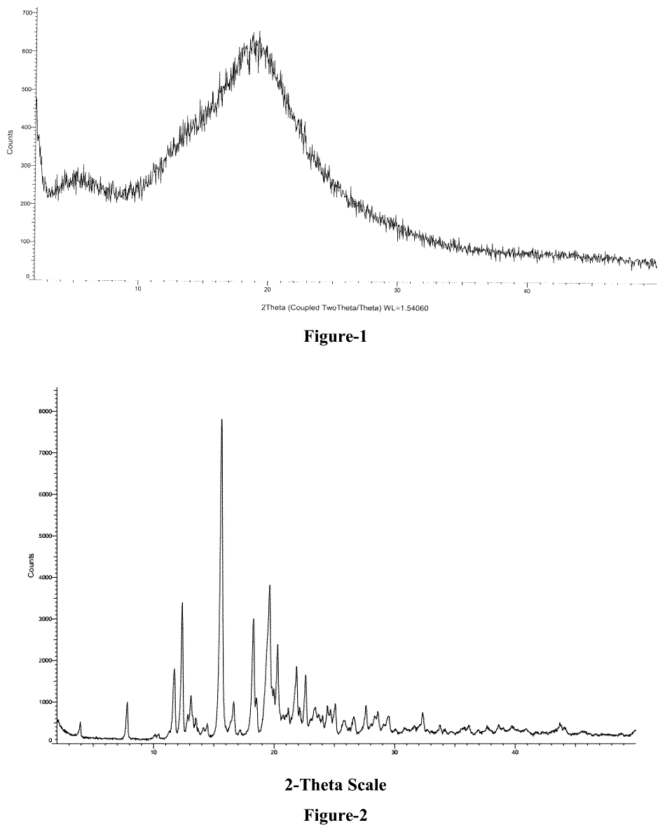

Yield: 15 g. PXRD of the obtained compound is depicted in FIG. 1.

1. A process for the preparation of Zanubrutinib of Formula-1, comprising: 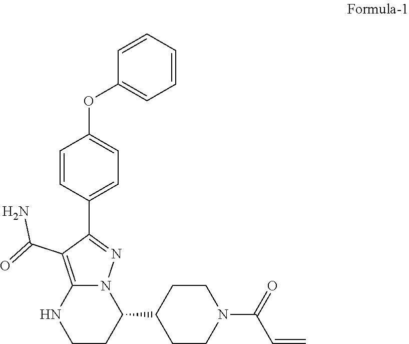 [SMILES: C=CC(=O)N1CCC([C@@H]2CCNc3c(C(N)=O)c(-c4ccc(Oc5ccccc5)cc4)nn32)CC1]
reacting the compound of Formula-3 with compound of Formula-4 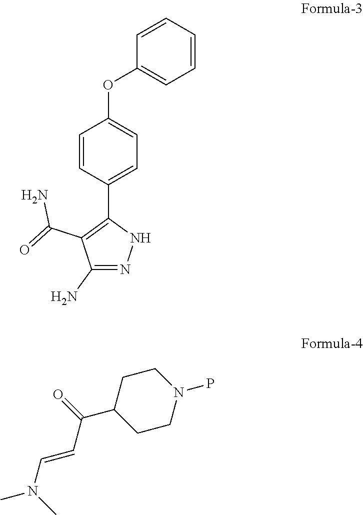
wherein P is amino protecting group;
to provide the compound of Formula-5

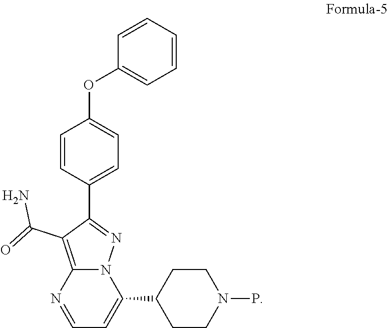

2. A process for the preparation of Zanubrutinib of Formula-1 comprising:
a) reduction of compound of Formula-5 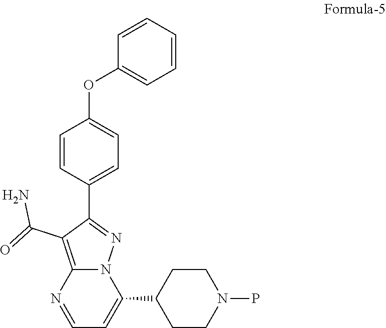
wherein P is amino protecting group;
using a reducing agent to provide compound of formula-6 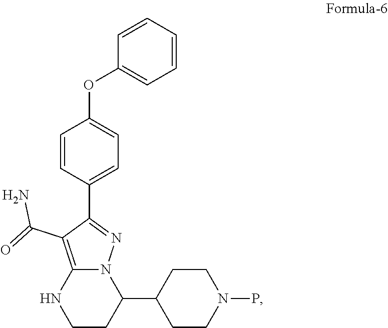
b) deprotecting the compound of Formula-6 using deprotecting agent to get compound of Formula-7a or salts thereof

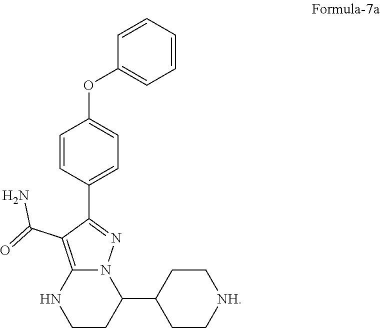 [SMILES: NC(=O)c1c(-c2ccc(Oc3ccccc3)cc2)nn2c1NCCC2C1CCNCC1]

3. The process as claimed in claim-1, wherein the reaction is carried out in a solvent selected from hydrocarbon solvents, ether solvents, ester solvents, chloro solvents, alcoholic solvents, polar aprotic solvents, ketone solvents, nitrile solvents, polar solvents, acetic acid or mixtures thereof.

4. The process as claimed in claim-2, wherein the reaction is carried out in a solvent selected from hydrocarbon solvents, ether solvents, ester solvents, chloro solvents, alcoholic solvents, polar aprotic solvents, ketone solvents, nitrile solvents, polar solvents, acetic acid or mixtures thereof.

5. The process as claimed in claim-1, wherein the reaction is carried out in presence of an acid selected from organic acids such as formic acid, acetic acid, propionic acid, oxalic acid, trifluoroacetic acid, methane sulfonic acid, p-toluenesulphonic acid and/or mixtures thereof and inorganic acid is selected from hydrochloric acid, hydrobromic acid, sulfuric acid, nitric acid and phosphoric acid.

6. (canceled)

7. The process as claimed in claim-2, wherein the reducing agent used in step-a) is selected from metal hydrides such as sodium borohydride, sodium cyanoborohydride, sodium triacetoxyborohydride, lithium borohydride, zinc borohydride or catalytic hydrogenation selected from Pd, Pt, Ni, Ru, Pd/C, Pt/C, Rh/C, Raney Ni, Pd(OH)₂/C, palladium acetate, platinum oxide hydrate and platinum black.

8. The process as claimed in claim-2, wherein the deprotecting agent is selected from hydrochloric acid, hydrobromic acid, methanesulfonic acid, ethanesulfonic acid, p-toluenesulfonic acid and/or in combination with alcohols.

9. The process as claimed in claim 2, the compound of formula-7a or salts thereof is further converting into Zanubrutinib of formula-1.

10-12. (canceled)

13. A process for the preparation of racemic compound of Formula-7 comprising:
a) reacting an enantiomerically enriched R-isomer compound of Formula-12 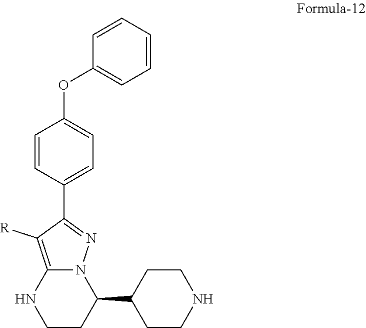
wherein R is CN, —CONH₂;
with an oxidizing agent in a solvent to provide a compound of Formula-13 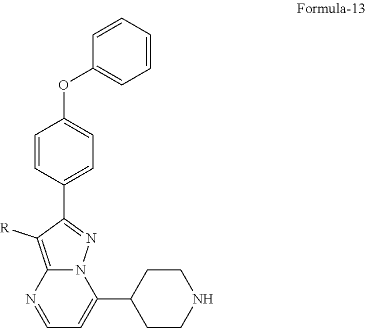
b) reducing the compound of Formula-13 in presence of reducing agent in a solvent to provide the racemic compound of Formula-7

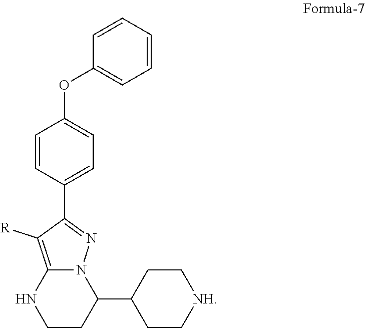

14. The process as claimed in claim 13, wherein the oxidizing agent used in step-a) is selected from manganese dioxide, potassium permanganate, 4,5-dichloro-3,6-dihydroxyphthalonitrile, peracetic acid, trifluoro peracetic acid, perbenzoic acid, and m-chloroperbenzoic acid.

15. The process as claimed in claim 13, wherein the reducing agent used in step-b) is selected from catalytic hydrogenation in presence of transition metals catalysts including but not limited to Ni, Pd, Pt, Rh, Re, Ru, Ir or LiAlH₄, NaAlH₄, NaBH₄, KBH₄, Aluminium hydride (AlH₃) and diisobutylaluminium hydride (DIBAL).

16. The process as claimed in claim 13, wherein the solvent is selected from chloro solvents, ether solvents, alcohol solvents, hydrocarbon solvents, polar solvents and/or mixtures thereof.

17. A pharmaceutical composition comprising Zanubrutinib obtained according to claim 1 and at least one pharmaceutically acceptable excipient.

18. A pharmaceutical composition for treating a mammal by administering a therapeutically effective amount of Zanubrutinib of formula-1 obtained according to claim 1 for treating adult patients with mantle cell lymphoma (MCL) who have received at least one prior therapy.

19. The process as claimed in claim 2, further comprising;
a) resolution of compound of formula-7a with chiral acid to provide compound of formula-8,
b) reacting compound of formula-8 with acrolyl chloride in presence of a base to provide Zanubrutinib of formula-1.

20. The process as claimed in claim 13 wherein an enantiomerically enriched R-isomer compound of Formula-12 is prepared by a process comprising:
a) distilling the solvent from the filtrate obtained in the resolution of racemic compound of formula-7 diasteromeric salt which contains enantiomerically enriched R-isomer of Formula-12 with a chiral acid,
b) treating the obtained compound in step-a) with a base;
c) isolating the enantiomerically enriched R-isomer compound of Formula-12.
